(19) United States
(12) Patent Application Publication
(10) Pub. No.: US 2002/0045606 A1
(43) Pub. Date: Apr. 18, 2002

(54) Esters of vitamin D3 and uses thereof

(76) Inventors: Satyanarayana G. Reddy, Barrington, RI (US); Gino John Sasso, Rutherford, NJ (US)

Correspondence Address: LAHIVE & COCKFIELD, 28 STATE STREET, BOSTON, MA, 02109, US

(21) Appl. No.: 09/728,933

(22) Filed: Dec. 1, 2000

Related U.S. Application Data
(60) Provisional application No. 60/168,588, filed on Dec. 2, 1999.

(51) Int. Cl. A61K 31/593; C07C 401/00

(52) U.S. Cl. 514/167; 552/653

(57) ABSTRACT

Analogs of vitamin D₃, in particular esters of vitamin D₃ and uses thereof, are described. The compounds of the present invention can be used as substitutes for natural and synthetic vitamin D₃ compounds.

### RELATED APPLICATIONS

This application claims priority to U.S. Provisional Patent Application Ser. No. 60/168,588, filed on Dec. 2, 1999, entitled “Esters of Vitamin D₃ Compounds and Uses Thereof.” The entire contents of this provisional application are hereby incorporated herein by reference. U.S. Pat. Nos. 6,017,908, 6,100,294, and 6,121,312 and U.S. patent application Ser. No. 09/080,026, filed May 15, 1998, relate to vitamin D technology and are hereby incorporated by reference in their entireties.

### BACKGROUND OF THE INVENTION

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D₃ [25(OH)D₃] (Blunt, J. W. et al. (1968) Biochemistry 6:3317-3322) and the hormonally active form, 1α,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1α,25(OH)₂D₃ in a carefully regulated fashion (Fraser, D. R. and Kodicek, E (1970) Nature 288:764-766; Wong, R. G. et al. (1972) J. Clin. Invest. 51:1287-1291), and the discovery of a nuclear receptor for 1α,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975). The operation of the vitamin D endocrine system depends on the following: first, on the presence of cytochrome P450 enzymes in the liver (Bergman, T. and Postlind, H. (1991) Biochem. J. 276:427-432; Ohyama, Y and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1α,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1α,25(OH)₂D₃ to generate the requisite specific biological responses for this secosteroid hormone (Pike, J. W. (1991) Annu. Rev. Nutr. 11:189-216). To date, there is evidence that nuclear receptors for 1α,25(OH)₂D₃ (VD₃R) exist in more than 30 tissues and cancer cell lines (Reichel, H. and Norman, A. W. (1989) Annu. Rev. Med. 40:71-78).

Vitamin D₃ and its hormonally active forms are well-known regulators of calcium and phosphorous homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1α,25(OH)₂ D₃ has been suggested by the combined presence of enzymes capable of oxidizing vitamin D₃ into its active forms, e.g., 25-OHD-1α-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40:71-78).

Given the pluripotent activities of vitamin D₃ and its metabolites, much attention has focused on the development of synthetic analogs of these compounds. A large number of these analogs involve structural modifications in the A ring, B ring, C/D rings, and, primarily, the side chain (Bouillon, R. et al. , Endocrine Reviews 16(2):201-204). Although a vast majority of the vitamin D₃ analogs developed to date involve structural modifications in the side chain, a few studies have reported the biological profile of A-ring diastereomers (Norman, A. W. et al. J. Biol. Chem. 268 (27):20022-20030). While biological esterification of steroids has been studied (Hochberg, R. B., (1998) Endocr Rev. 19(3):331-348), and esters of vitamin D₃ are known (WO 97/11053), there remains a need for vitamin D₃ analogs which exhibit sustained release, without diminished potency efficacy, or cell specificity. Moreover, despite much effort in developing synthetic analogs, clinical applications of vitamin D₃ and its structural analogs have been limited by the undesired side effects elicited by these compounds after administration to a subject, such as the deregulation of calcium and phosphorous homeostasis in vivo that results in hypercalcemia.

### SUMMARY OF THE INVENTION

The present invention is based, at least in part, on the discovery of vitamin D₃ ester compounds, in particular fatty acid ester compounds, such as those represented by formula I infra. The vitamin D₃ fatty acid ester compounds of the present invention can be produced in vitro via a metabolic pathway in certain specific tissues, e.g., vascular smooth cells and bone cells. The vitamin D₃ ester compounds of the present invention can be used as substitutes for natural and synthetic forms of vitamin D₃.

Accordingly, the present invention pertains to isolated compounds represented by the formula (Formula I):

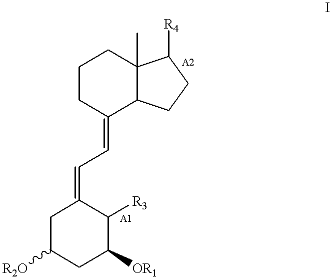

wherein A₁ is a single or double bond; A₂ is a single bond or a double bond; R₁ and R₂ are each hydrogen or a hydrolyzable moiety, provided that R₁ and R₂ are not both hydrogen; R₃ is hydrogen, deuteriumn, deuteroalkyl, hydroxyl, alkyl, alkoxide, O-acyl, halogen, haloalkyl, hydroxyalkyl, amino or thiol; and R₄ is a saturated or unsaturated carbon chain represented by the formula:

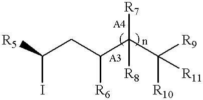

wherein I represents the above-described formula I; A₃ and A₄ are each, independently, a single bond or a double bond; R₅, R₆, R₇, and R₈, are each, independently, hydrogen, deuterium, hydroxyl, alkyl, alkoxide, O-acyl, halogen, haloalkyl, hydroxyalkyl, oxygen, amino or thiol; R₉ and R₁₀ are each, independently, alkyl, hydroxyalkyl, halogen, hydroxyl, haloalkyl or deuteroalkyl; R₁₁ is hydrogen, hydroxyl or O-acyl; and n is an integer from 1 to 5.

In one embodiment, the isolated form of a vitamin D₃ compound of the invention has formula I wherein A₁ is a double bond, A₂, A₃ and A₄ are single bonds, R₆, R₇ and R₈ are hydrogen, R₅, R₉ and R₁₀ are methyl, n is 1, and the substituent R₂O at the 3-carbon position is in the, β-configuration.

In another embodiment, the invention pertains to an isolated compound represented by the formula (Formula II):

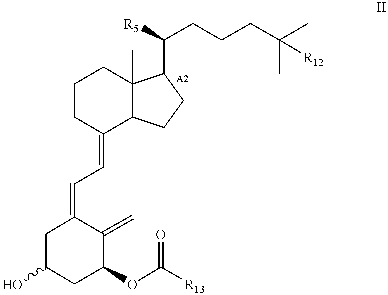

wherein A₂ is a single bond or a double bond; R₅ is deuterium, hydroxyl, alkyl, alkoxide, O-acyl, halogen, haloalkyl, hydroxyalkyl, oxygen, amino or thiol; R₁₂ is hydrogen, hydroxyl or O-acyl; and R₁₃ is C₁-C₂₆ alkyl, aryl or aralkyl.

In another embodiment, the invention pertains to an isolated compound represented by the formula (Formula III):

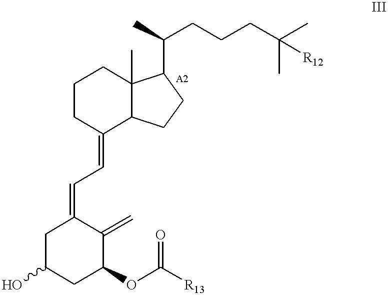

wherein A₂ is a single bond or a double bond; R₁₂ is hydrogen or hydroxyl; and R₁₃ is a side chain of a naturally occurring fatty acid.

In another aspect, the present invention further pertains to a pharmaceutical composition comprising, a therapeutically effective amount of a compound represented by formula I, II or III and a pharmaceutically acceptable carrier.

In yet another aspect, the invention provides a method of modulating a biological activity of a vitamin D₃-responsive cell. This method comprises contacting the cell with an effective amount of a compound of formula I, II or III such that modulation of the activity of the cell occurs.

Another aspect of the invention provides a method of treating in a subject a disorder characterized by aberrant growth or activity of a cell, comprising administering to the subject an effective amount of a compound of formula I, II or III such that the growth or activity of the cell is reduced. In one embodiment, the subject is a mammal. In a preferred embodiment, the subject is human.

In a preferred embodiment, the compound of formula I, II or III used in the treatment has improved biological properties compared to vitamin D₃, such as enhanced stability and/or reduced toxicity.

In one aspect, a method for inhibiting the proliferation and/or an inducing the differentiation of a hyperproliferative skin cell is provided, wherein the hyperproliferative skin cell can be an epidermal cell or an epithelial cell. Accordingly, therapeutic methods for treating hyperproliferative skin disorders, e.g., psoriasis, are provided.

In certain embodiments, the instant method can be used for the treatment of, or prophylactic prevention of a disorder characterized by aberrant cell growth of vitamin D₃-responsive neoplastic cell, e.g., by administering a pharmaceutical preparation of a compound having the formula I, II or III in an amount effective to inhibit growth of the neoplastic cells.

In yet another aspect, the compounds of the present invention are useful in the treatment of disorders characterized by a deregulation of calcium and phosphate metabolism, comprising administering to a subject a pharmaceutical preparation of a compound of formula I, II or III so as to ameliorate the deregulation in calcium and phosphate metabolism.

In a preferred embodiment, the disorder is osteoporosis. In other embodiments, the compounds of formula I, II or III can be used to treat diseases characterized by other deregulations in the metabolism of calcium and phosphate.

In another aspect, a method for inhibiting PTH secretion in parathyroid cell using the compounds of formula I, II or III is provided. Furthermore, therapeutic methods for treating secondary hyperparathyroidism are also provided.

In yet another aspect, the present invention provides a method of preventing or protecting against neuronal loss by contacting a vitamin D₃-responsive cell, e.g., a neuronal cell, with a compound of formula I, II or III to prevent or retard neuron loss.

In yet another aspect, the present invention provides a method of modulating the activity of a vascular smooth muscle cell by contacting a vitamin D₃-responsive smooth muscle cell with a compound of formula I, II or III to activate or, preferably, inhibit the activity of the cell.

In still another aspect, the present invention provides a packaged compound comprising a compound of formula I, II or III with instructions for use of the compound for treating a disorder characterized by an aberrant activity of a vitamin D₃-responsive cell.

### BRIEF DESCRIPTION OF THE DRAWINGS

FIG. 1 shows the HPLC spectra of metabolites of 1α,25(OH)₂-16-ene-D₃, 1α,25(OH)₂-16-ene-3-epi-D₃, 1α,25(OH)₂-16-ene-20-epi-D₃, 1α,25(OH)₂-16-ene-20-epi-3-epi-D₃, and 1α,25(OH)₂-16-ene-23-yne-D₃, using HPLC system I, as described in Example II.

FIG. 2 shows the HPLC spectra of metabolites of 1α,25(OH)₂-16-ene-D₃, 1α,25(OH)₂-16-ene-3-epi-D₃, 1α,25(OH)₂-16-ene-20-epi-D₃, 1α,25(OH)₂-16ene 20-epi-3-epi-D₃, and 1α,25(OH)₂-16-ene-23-yne-D₃, using HPLC system II, as described in Example II.

### DETAILED DESCRIPTION OF THE INVENTION

The present invention relates to isolated analogs of vitamin D₃, as well as methods of treating disorders characterized by aberrant activity of a vitamin D₃ responsive cell. The compounds of the invention are effective therapeutic agents for such conditions as osteoporosis (including senile osteoporosis and post-menapausal osteoporosis), osteodystrophy, osteomalacia, rickets, osteitis fibrosa cystica, renal osteodystrophy, secondary hyperparathyrodism, cirrhosis, and chronic renal disease. In particular, the invention provides compounds that exhibit the biological activity of vitamin D₃, in a “pro-drug” form that allows for sustained release in vivo activity of the compounds of the invention. In addition, the compounds of the invention may exhibit reduced toxicity and increased potency, as compared to other pro-drugs.

The invention provides compounds and methods which exploit the biological activity of vitamin D while also providing gradual onset or prolonged duration of this activity. This aspect of the invention is provided, in part, by the hydrolyzable moiety or moieties of the compounds disclosed herein. In preferred embodiments, the hydrolyzable moiety is an ester functional group, i.e. an O-acyl group. In particularly preferred embodiments, the hydrolyzable moiety includes a side chain of a fatty acid. The release rate of compounds of the invention may be varied or controlled depending on a variety of criteria, such as the type, size and structural complexity of the hydrolyzable moiety. This release rate may be further modified or controlled by combining a plurality of different compounds of the invention, or by combining one or more compounds of the invention with vitamin D, or vitamin D analogs. Without being bound by any theory, it is believed that the presence of hydrolyzable moieties at either or both of C₁ and C₃ of the compounds herein imparts the advantageous sustained release effect of these compounds.

1α,25(OH)₂D₃ is a hormonally active secosteroid. The term “secosteroid” is art-recognized and includes compounds in which one of the cyclopentanoperhydro-phenanthrene rings of the steroid ring structure is broken. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1α,25(OH)₂D₃ is illustrated herein having all carbon atoms numbered using standard steroid notation.

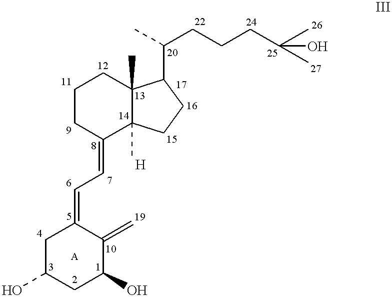

In the formulas presented herein, the various substituents are illustrated as joined to the steroid nucleus by one of these notations: a dotted line (______) indicating a substituent which is in the β-orientation (i.e., above the plane of the ring), a wedged solid line ( [symbol] ) indicating a substituent which is in the ax-orientation (i.e. , below the plane of the molecule), or a wavy line ( [symbol] ) indicating that a substituent may be either above or below the plane of the ring. It should be understood that the stereochemical convention in the vitamin D field is opposite from the general chemical field, wherein a dotted line indicates a substituent which is in an α-orientation (i.e. , below the plane of the molecule), and a wedged solid line indicates a substituent which is in the β-orientation (i.e., above the plane of the ring). As shown, the A ring of the hormone 1α,25(OH)₂D₃ contains two asymmetric centers at carbons 1 and 3, each one containing a hydroxyl group in well-characterized configurations, namely the 1α- and 3β-hydroxyl groups. In other words, carbons 1 and 3 of the A ring are said to be “chiral carbons” or “carbon centers.”

With respect to the nomenclature of a chiral center, terms “d” and “I” configuration are as defined by the IUPAC Recommendations. As to the use of the terms, diastereomer, racemate, epimer and enantiomer will be used in their normal context to describe the stereochemistry of preparations.

In one embodiment, the invention provides compounds of formula I wherein the substituent at the 1-carbon position is in the α-configuration. In another embodiment, the substituent at the 3-carbon is in the β-configuration. In preferred embodiments, the invention provides compounds of formula I or II wherein R₂ is hydrogen. In other embodiments, the invention provides compounds of formula I or II wherein A₁ is a double bond. In some embodiments, the invention provides compounds of formula I or II wherein R₃ is methyl. In other embodiments, the invention provides compounds of formula I or II wherein R₅ is methyl and R6 is hydrogen; in more preferred embodiments, the methyl is in the α-configuration. In still other embodiments, the invention provides compounds of formula I, II, III or IV wherein A₂ is a double bond. In other preferred embodiments, the invention provides compounds of formula I, II, III or IV wherein R₁₂ is hydroxyl or hydrogen. In a preferred embodiment, R₁₂ is hydroxyl. In yet another preferred embodiment, the invention provides compounds of formula I or II wherein R₁ has the formula —C(═O)R₁₃ wherein R₁₃ is C₁-C₂₆ alkyl, aryl or aralkyl.

In some embodiments, the invention provides compounds of formula I, II, III or IV wherein R₁₃ has the formula —(CH₂)ₓ—CH═CH—(CH₂)_{y}—CH₃, wherein x and y are an integer from 1 to 10; in other embodiments, R₁₃ has the formula —(CH₂)_{z}CH₃, wherein z is an integer from 1 to 25. In preferred embodiments, the invention provides compounds of formula I, II, III, or IV wherein R₁₃ is a side chain of a fatty acid; more preferably, R,₁₃ is a side chain of a naturally occurring fatty acid; even more preferably, R₁₃ is a side chain of lauric acid, myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, or cerebronic acid. In a particularly preferred embodiment, R₁₃ is the side chain of stearic acid or oleic acid. Preferred compounds include 3-epi-25-hydroxy-16-ene-20-epi-D₃-1-α-stearate, 3-epi-25-hydroxy-16-ene-20-epi-D₃-1-α-oleate, 25-hydroxy-16-ene-20-epi-D₃-1-α-stearate, 25-hydroxy-16-ene-20-epi-D₃-1-α-oleate, 3-epi-25-hydroxy-20-epi-D₃-1-α-stearate, 3-epi-25-hydroxy-20-epi-D₃-1-α-oleate, 25-hydroxy-20-epi-D₃-1-α-stearat and 25-hydroxy-20-epi-D₃-1-α-oleate.

In one embodiment, the invention provides compounds of formula III or IV wherein the hydroxyl group at the 3-position is in the α-configuration. In another embodiment, the invention provides compounds of formula III or IV wherein the hydroxyl group at the 3-postion is in the β-configuration.

In yet another aspect, the invention provides a method of treating a disorder characterized by an aberrant activity of a vitamin D₃-responsive cell, comprising administering to a subject an effective amount of a compound of formula I, II or III, such that the aberrant activity of the vitamin D₃-responsive cell is reduced.

### Definitions

So that the present invention may be more readily understood, a number of pertinent terms are first defined.

The term “administration,” is intended to include routes of introducing a compound of the invention to perform their intended function. Examples of routes of administration which can be used include injection (subcutaneous, intravenous, parenterally, intraperitoneally, intrathecal, etc.), oral, inhalation, rectal and transdermal (e.g., topical). The pharmaceutical preparations are of course given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally effect its ability to perform its intended function. The compound of the invention can be administered alone, or in conjunction with either another agent as described above or with a pharmaceutically acceptable carrier, or both. The compound of the invention can be administered prior to the administration of the other agent, simultaneously with the agent, or after the administration of the agent. Furthermore, the compound of the invention can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g. , phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g. , tetralin).

The terms “alkenyl” and “alkynyl” refer to unsaturated aliphatic groups analogous in length and possible substitution to the alkyls described above, but that contain at least one double or triple bond respectively. For example, the invention contemplates cyano and propargyl groups.

The language “biological activities” of vitamin D₃ is intended to include all activities elicited by vitamin D₃ compounds in a responsive cell. This term includes genomic and non-genomic activities elicited by these compounds (Gniadecki R. and Calverley M. J. (1998) Pharmacology & Toxicology 82:173-176; Bouillon, R. et al. (1995) Endocrinology Reviews 16(2):206-207; Norman A. W. et al. (1992) J. Steroid Biochem Mol. Biol 41:231-240; Baran D. T. et al. (1991) J. Bone Miner Res. 6:1269-1275; Caffrey J. M. and Farach-Carson M. C. (1989) J. Biol. Chem. 264:20265-20274; Nemere I. et al. (1984) Endocrinology 115:1476-1483).

The language “bone metabolism” is intended to include direct or indirect effects in the formation or degeneration of bone structures, e.g., bone formation, bone resorption, etc., which may ultimately affect the concentrations in serum of calcium and phosphate. This term is also intended to include effects of compounds of the invention in bone cells, e.g., osteoclasts and osteoblasts, that may in turn result in bone formation and degeneration.

As used herein, the term “calcium and phosphate homeostasis” refers to the careful balance of calcium and phosphate concentrations, intracellularly and extracellularly, triggered by fluctuations in the calcium and phosphate concentration in a cell, a tissue, an organ or a system. Fluctuations in calcium levels that result from direct or indirect responses to compounds of the invention are intended to be included by these terms.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “epimer” or “epi compounds” is intended to include compounds having a chiral carbon that varies in the orientation of a single bond to a substituent on that carbon compared to the naturally-occurring (or reference) compound; for example, a carbon where the orientation of the bond to the substituent is in an α-configuration, instead of a β-configuration. The 3-epimer form of vitamin D₃ having the general formula I has a hydroxyl group attached to the carbon at position 3 of the A-ring in an α-configuration rather than a β-configuration, whereas all other substituents can be in either an α- or a β-configuration.

The term “esterase cleavable moiety” refers to a substituent which may be removed from a molecule by the enzyme esterase, under conditions known in the art.

The term “fatty acid” is art-recognized and refers to the class of carbohydrates containing a terminal carboxyl group and a carbon chain or “side chain” of at least ten carbon atoms. The fatty acid esters of the present invention are esters of fatty acids which preferably have between 14 and 22 carbon atoms in the side chain, more preferably 16 to 18 carbon atoms in the side chain. The side chain of fatty acids encompassed by the present invention may be saturated or unsaturated, and linear or cyclic. In addition, fatty acid side chains encompassed by this invention may be substituted or unsubstituted, and may contain heteroatoms, such as nitrogen, oxygen, or sulfur. Some preferred naturally occurring fatty acids are listed in Table 1.

TABLE 1
Some Naturally Occurring Fatty Acids

| STRUCTURE | COMMON NAME |
|---|---|
| CH₃(CH₂)₁₀COOH | Lauric acid |
| CH₃(CH₂)₁₂COOH | Myristic acid |
| CH₃(CH₂)₁₄COOH | Palmitic acid |
| CH₃(CH₂)₁₆COOH | Stearic acid |
| CH₃(CH₂)₁₈COOH | Arachidic acid |
| CH₃(CH₂)₂₂COOH | Lignoceric acid |
| CH₃(CH₂)₅CH═CH(CH₂)₇COOH | Palmitoleic acid |
| CH₃(CH₂)₇CH═CH(CH₂)₇COOH | Oleic acid |
| CH₃(CH₂)₄CH═CHCH₂CH═CH(CH₂)₇COOH | Linoleic acid |
| CH₃CH₂CH═CHCH₂CH═CHCH₂CH═ | Linolenic acid |
| CH(CH₂)₇COOH | |
| CH₃(CH₂)₄(CH═CHCH₂)₃CH═ | Arachidonic |
| CH(CH₂)₃COOH | |
| CH₃(CH₂)₅CH═CH(CH₂)₇COOH(trans) | trans-Hexadecanoic |
| CH₃(CH₂)₇CH═CH(CH₂)₇COOH(trans) | Elaidic acid |
| 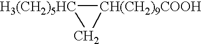 | Lactobacillic acid |
| 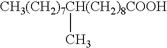 | Tuberculostearic acid |
| 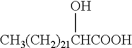 | Cerebronic acid |

The language “genomic” activities or effects of vitamin D₃ is intended to include those activities mediated by the nuclear receptor for 1 α,25(OH)₂D₃ (VD₃R), e.g., transcriptional activation of target genes.

As used herein, the term “halogen” designates —F, —Cl, —Br or —I; the term “sulfhydryl” or “thiol” means —SH; the term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “hormone secretion” is art-recognized and includes activities of vitamin D₃ compounds that control the transcription and processing responsible for secretion of a given hormone e.g., parathyroid hormone (PTH) a vitamin D₃ responsive cell (Bouillon, R. et al. (1995) Endocrine Reviews 16(2):235-237).

The term “hydrolyzable moiety” as used herein refers to any substituent which may be removed by the process of hydrolysis, preferably in vivo hydrolysis, e.g., by an esterase. Preferred embodiments include, but are not limited to, an ester functional group, i.e. an O-acyl group. In particularly preferred embodiments, the hydrolyzable moiety includes a side chain of a fatty acid. In most preferred embodiments, the hydrolyzable moiety includes a side chain of a naturally occuring fatty acid.

The language “hypercalcemia” or “hypercalcemic activity” is intended to have its accepted clinical meaning, namely, increases in calcium serum levels that are manifested in a subject by the following side effects, depression of central and peripheral nervous system, muscular weakness, constipation, abdominal pain, lack of appetite and, depressed relaxation of the heart during diastole. Symptomatic manifestations of hypercalcemia are triggered by a stimulation of at least one of the following activities, intestinal calcium transport, bone calcium metabolism and osteocalcin synthesis (reviewed in Boullion, R. et al. (1995) Endocrinology Reviews 16(2):200-257).

As used herein, the language “improved biological properties” refers to any activity inherent in a compound of the invention that enhances its effectiveness in vivo. In a preferred embodiment, this term refers to any qualitative or quantitative improved therapeutic property of a vitamin D₃ compound, such as enhanced stability in vivo and/or reduced toxicity, e.g., reduced hypercalcemic activity.

The term “isomers ” or “stereoisomers” refers to compounds which have identical chemical constitution, but differ with regard to the arrangement of the atoms or groups in space.

The terms “isolated” or “substantially purified” are used interchangeably herein and refer to vitamin D₃ compounds in a non-naturally occurring state. The compounds can be substantially free of cellular material or culture medium when naturally produced, or chemical precursors or other chemicals when chemically synthesized. In certain preferred embodiments, the terms “isolated” or “substantially purified” also refer to preparations of a chiral compound which substantially lack one of the enantiomers; i.e., enantiomerically enriched or non-racemic preparations of a molecule. Similarly, the terms “isolated epimers” or “isolated diastereomers” refer to preparations of chiral compounds which are substantially free of other stereochemical forms. For instance, isolated or substantially purified vitamin D₃ compounds include synthetic or natural preparations of a vitamin D₃ enriched for the stereoisomers having a substituent attached to the chiral carbon at position 3 of the A-ring in an α-configuration, and thus substantially lacking other isomers having a β-configuration. Unless otherwise specified, such terms refer to vitamin D₃ compositions in which the ratio of α to β forms is greater than 1:1 by weight. For instance, an isolated preparation of an a epimer means a preparation having greater than 50% by weight of the α-epimer relative to the β stereoisomer, more preferably at least 75% by weight, and even more preferably at least 85% by weight. Of course the enrichment can be much greater than 85%, providing “substantially epimer-enriched” preparations, i.e., preparations of a compound which have greater than 90% of the α-epimer relative to the β stereoisomer, and even more preferably greater than 95%. The term “substantially free of the β0 stereoisomer” will be understood to have similar purity ranges.

As used herein, the language “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g. a therapeutic result. In preferred embodiments, this phrase is intended to include hyperactive conditions that result in pathological disorders.

The language “non-genomic” vitamin D₃ activities include cellular (e.g. calcium transport across a tissue) and subcellular activities (e.g., membrane calcium transport opening of voltage-gated calcium channels, changes in intracellular second messengers) elicited by vitamin D₃ compounds in a responsive cell. Electrophysiological and biochemical techniques for detecting these activities are known in the art. An example of a particular well-studied non-genomic activity is the rapid hormonal stimulation of intestinal calcium mobilization, termed “transcaltachia” (Nemere I. et al. (1984) Endocrinology 115:1476-1483; Lieberherr M. et al. (1989) J. Biol. Chem. 264:20403-20406; Wali R. K. et al. (1992) Endocrinology 131:1125-1133; Wali R. K. et al. (1992) Am. J. Physiol. 262:G945-G953; Wali R. K. et al. (1990) J. Clin. Invest. 85:1296-1303; Bolt M. J. G. et al. (1993) Biochem. J. 292:271-276). Detailed descriptions of experimental transcaltachia are provided in Norman, A. W. (1993) Endocrinology 268(27):20022-20030; Yoshimoto, Y. and Norman, A. W. (1986) Endocrinologyl 18:2300-2304. Changes in calcium activity and second messenger systems are well known in the art and are extensively reviewed in Bouillion, R. et al. (1995) Endocrinology Review 16(2):200-257; the description of which is incorporated herein by reference.

The phrase “pharmaceutically acceptable” is employed herein to refer to those vitamin D₃ ester compounds of the invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (1₃) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The phrase “pharmaceutically acceptable” is employed herein to refer to those vitamin D₃ ester compounds of formula I, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling.

The language “reduced toxicity” is intended to include a reduction in any undesired side effect elicited by a vitamin D₃ compound when administered in vivo, e.g., a reduction in the hypercalcemic activity.

The phrases “systemic administration,” “administered systemically,” “peripheral administration” and “administered peripherally,” as used herein, mean the administration of a compound(s) of the invention, drug or other material, such that it enters the patient's system and, thus, is subject to metabolism and other like processes, for example, subcutaneous administration.

The phrase “therapeutically-effective amount” as used herein means that amount of a compound(s) of the invention, or composition comprising such a compound which is effective for the compound to produce its intended function, e.g., the modulation of activity of a vitamin D₃-response cell. The effective amount can vary depending on such factors as the type of cell growth being treated or inhibited, the particular type of compound of the invention, the size of the subject, or the severity of the undesirable cell growth or activity. One of ordinary skill in the art would be able to study the aforementioned factors and make the determination regarding the effective amount of the vitamin D₃ fatty acid ester compound of the invention without undue experimentation.

The term “VD₃Rs” is intended to include members of the type II class of steroid/thyroid superfamily of receptors (Stunnenberg, H. G. (1993) Bio Essays 15(5):309-15), which are able to bind transactivate through the vitamin D response element (VDRE) in the absence of a ligand (Damm et al. (1989) Nature 339:593-97; Sap et al. Nature 343:177-180).

As used herein “VDREs” refer to a DNA sequences composed of half-sites arranged as direct repeats. It is known in the art that type II receptors do not bind to their respective binding site as homodimers but require an auxiliary factor, RXR (e.g. RXRα, RXRβ, RXRγ) for high affinity binding Yu et al. (1991) Cell 67:1251-1266; Bugge et al.(1992) EMBO J. 11:1409-1418; Kliewer et al. (1992) Nature 355:446-449; Leid et al. (1992) EMBO J. 11:1419-1435; Zhang et al. (1992) Nature 355:441-446).

The language “vitamin D₃ responsive cells” as used herein is intended to include endocrine cells which respond to compounds of the invention by altering gene expression and/or post-transcriptional processing secretion of a hormone.

It will be noted that the structure of some of the compounds of the invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### Synthesis of Compounds of the Invention

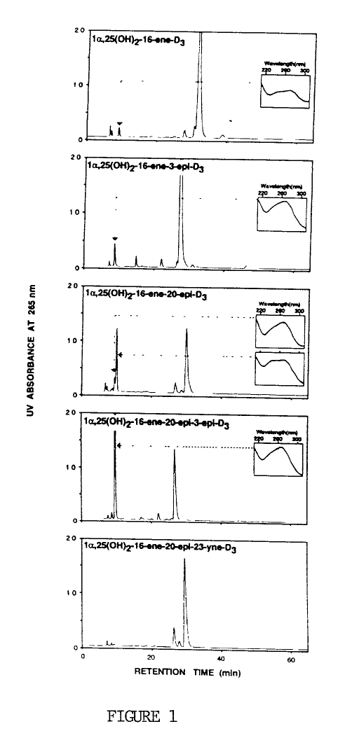

The compounds of the present invention can be prepared by incubation of vitamin D₃ analogs in cells. As described in the examples, incubation of vitamin D₃ analogs in either UMR 106 cells or Ros 17/2.8 cells results in production of vitamin D₃ fatty acid ester compounds of the invention. As shown in FIG. 1, incubation of 1α,25(OH)₂-16-ene-D₃ in UMR 106 cells results in production of the less polar fatty acid ester metabolites. Incubation of the 3α epimer, namely 1α,25(OH)₂-16-ene-3-epi-D₃, results in slightly greater amounts of one of the less polar metabolites. However, when 1α,25(OH)₂-16-ene-20-epi-D₃ is used, a greater increase in the amount of the less polar fatty acid ester metabolites results. This production is enhanced even further when 1α,25(OH)₂-16-ene-20-epi-3-epi-D₃ is incubated in UMR 106 cells. In contrast, when 1α,25(OH)₂-16-ene-23-yne-D₃ was incubated in UMR 106 cells, the amount of less polar fatty acid ester metabolites produced is reduced.

In addition to the foregoing methods, compounds of the present invention can be prepared using a variety of synthetic methods. For example, methods for synthesizing compounds of the invention are well known in the art (see e.g., Bouillon, R. et al., Endocrine Reviews 16(2):201-204; Ikekawa N. (1987) Med. Res. Rev. 7:333-366; DeLuca H. F. and Ostrem V. K. (1988) Prog. Clin. Biol. Res. 259:41-55; Ikekawa N. and Ishizuka S. (1992) CRC Press 8:293-316; Calverley M. J. and Jones G. (1992) Academic Press 193-270; Pardo R. and Santelli M. (1985) Bull. Soc. Chim. Fr:98-114; Bythgoe B. (1980) Chem. Soc. Rev. 449-475; Quinkert G. (1985) Synform 3:41-122; Quinkert G. (1986) Synform 4:131-256; Quinkert G. (1987) Synform 5:1-85; Mathieu C. et al. (1994) Diabetologia 37:552-558; Dai H. and Posner G. H. (1994) Synthesis 1383-1398); DeLuca et al., WO 97/11053. Exemplary methods of synthesis include the photochemical ring opening of a 1-hydroxylated side chain-modified derivative of 7-dehydrocholesterol which initially produces a previtamin that is easily thermolyzed to vitamin D₃ in a well known fashion (Barton D. H. R. et al. (1973) J. Am. Chem. Soc. 95:2748-2749; Barton D. H. R. (1974) JCS Chem. Comm. 203-204); phosphine oxide coupling method developed by (Lythgoe et al.( 1978) JCS Perkin Trans. 1:590-595) which comprises coupling a phosphine oxide to a Grundmann's ketone derivative to directly produce a 1α,25(OH)₂D₃ skeleton as described in Baggiolini E. G. et al. (1986) J. Org. Chem. 51:3098-3108; DeSchrijver J. and DeClercq P. J. (1993) Tetrahed Lett 34:4369-4372; Posner G. H. and Kinter C. M. (1990) J. Org. Chem. 55:3967-3969; semihydrogenation of dienynes to a previtamin structure that undergoes rearrangement to the corresponding vitamin D₃ analog as described by Harrison R. G. et al. (1974) JCS Perkin Trans. 1:2654-2657; Castedo L. et al. (1988) Tetrahed Lett 29:1203-1206; Mascarenas J. S. (1991) Tetrahedron 47:3485-3498; Barrack S. A. et al. (1988) J. Org. Chem. 53:1790-1796) and Okamura W. H. et al. (1989) J. Org. Chem. 54:4072-4083; the vinylallene approach involving intermediates that are subsequently arranged using heat or a combination of metal catalyzed isomerization followed by sensitized photoisomerization (Okamura W. H. et al. (1989) J. Org. Chem. 54:4072-4083; Van Alstyne E. M. et al. (1994) J. Am. Chem. Soc. 116:6207-6210); the method described by Trost et al. B. M. et al. J. Am. Chem. Soc. 114:9836-9845; Nagasawa K. et al. (1991) Tetrahed Lett 32:4937-4940 involves an acyclic A-ring precursor which is intramolecular cross-coupled to the bromoenyne leading directly to the formation of 1,25(OH)₂D₃ skeleton; a tosylated derivative which is isomerized to the i-steroid that can be modified at carbon-1 and then subsequently back-isomerized under sovolytic conditions to form 1α,25(OH)₂D₂ or analogs thereof (Sheves M. and Mazur Y. (1974) J. Am. Chem. Soc. 97:6249-6250; Paaren H. E. et al. (1980) J. Org Chem. 45:3253-3258; Kabat M. et al. (1991) Tetrahed Lett 32:2343-2346; Wilson S. R. et al. (1991) Tetrahed Lett 32:2339-2342); the direct modification of vitamin D derivatives to 1-oxygenated 5, 6-trans vitamin D as described in (Andrews D. R. et al. (1986) J. Org. Chem. 51:1635-1637); the Diels-Alders cycloadduct method of previtamin D₃ can be used to cyclorevert to 1α,25(OH)₂D₂ through the intermediary of a previtamin form via thermal isomerization (Vanmaele L. et al. (1985) Tetrahedron 41:141-144); and, a final method entails the direct modification of 1α,25(OH)₂D₂ or an analog through use of suitable protecting groups such as transition metal derivatives or by other chemical transformations (Okarmura W. H. et al. (1992) J. Cell Biochem. 49:10-18). Additional methods for synthesizing vitamins D2 compounds are described in, for example, Japanese Patent Disclosures Nos. 62750/73, 26858/76, 26859/76, and 71456/77; U.S. Pat. Nos. 3,639,596; 3,715,374; 3,847,955 and 3,739,001.

Examples of the compounds of this invention having a saturated side chain can be prepared according to the general process illustrated and described in U.S. Pat. No. 4,927,815. Examples of the compounds of this invention having an unsaturated side chain is can be prepared according to the general process illustrated and described in U.S. Pat. No. 4,847,012. Examples of the compounds of this invention wherein R groups together represent a cyclopentano group can be prepared according to the general process illustrated and described in U.S. Pat. No. 4,851,401.

Another synthetic strategy for the preparation of side-chain-modified analogues of 1α,25-dihydroxyergocalciferol is disclosed in Kutner et al., The Journal of Organic Chemistry, 1988, 53:3450-3457. In addition, the preparation of 24-homo and 26-homo vitamin D analogs are disclosed in U.S. Pat. No. 4,717,721.

The enantioselective synthesis of chiral molecules is now state of the art. Through combinations of enantioselective synthesis and purification techniques, many chiral molecules can be synthesized as an enantiomerically enriched preparation. For example, methods have been reported for the enantioselective synthesis of A-ring diastereomers of 1α,25(OH)₂D₃ as described in Muralidharan et al. (1993) J. Organic Chem. 58(7):1895-1899 and Norman et al. (1993) J. Biol. Chem. 268(27):20022-30. Other methods for the enantiomeric synthesis of various compounds known in the art include, inter alia, epoxides (see, e.g., Johnson, R. A.; Sharpless, K. B. In Catalytic Asymmetric Synthesis; Ojima, I., Ed.: VCH: New York, 1993; Chapter 4.1. Jacobsen, E. N. Ibid. Chapter 4.2), diols (e.g., by the method of Sharpless, J. Org. Chem. (1992) 57:2768), and alcohols (e.g., by reduction of ketones, E. J. Corey et al., J. Am. Chem. Soc. (1987) 109:5551). Other reactions useful for generating optically enriched products include hydrogenation of olefins (e.g., M. Kitamura et al., J. Org. Chem. (1988) 53:708); Diels-Alder reactions (e.g., K. Narasaka et al., J. Am. Chem. Soc. (1989) 111:5340); aldol reactions and alkylation of enolates (see, e.g., D. A. Evans et al., J. Am. Chem. Soc. (1981) 103:2127; D. A. Evans et al., J. Am. Chem. Soc. (1982) 104:1737); carbonyl additions (e.g., R. Noyori, Angew. Chem. Int. Ed. Eng. (1991) 30:49); and ring-opening of meso-epoxides (e.g., Martinez, L. E.; Leighton J. L., Carsten, D. H.; Jacobsen, E. N. J. Am. Chem. Soc. (1995) 117:5897-5898). The use of enzymes to produce optically enriched products is also well known in the art (e.g., M. P. Scheider, ed. “Enzymes as Catalysts in Organic Synthesis”, D. Reidel, Dordrecht (1986).

Chiral synthesis can result in products of high stereoisomer purity. However, in some cases, the stereoisomer purity of the product is not sufficiently high. The skilled artisan will appreciate that the separation methods described herein can be used to further enhance the stereoisomer purity of the vitamin D₃-epimer obtained by chiral synthesis.

### Pharmaceutical Compositions

In another aspect, the present invention provides pharmaceutically acceptable compositions which comprise a therapeutically-effective amount of one or more of the compounds of the invention, formulated together with one or more pharmaceutically acceptable carrier(s).

In a preferred embodiment, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In certain embodiments, the subject is a mammal, e.g., a primate, e.g., a human. As used herein, the language “subject” is intended to include human and non-human animals. Preferred human animals include a human patient having a disorder characterized by the aberrant activity of a vitamin D₃-responsive cell. The term “non-human animals” of the invention includes all vertebrates, e.g., mammals and non-mammals, such as non-human primates, sheep, dog, cow, chickens, amphibians, reptiles, etc.

In certain embodiments, one or more compounds of the invention may be administered alone, or as part of combinatorial therapy. For example, compounds of the invention can be conjointly administered with one or more agents such as mitotic inhibitors, alkylating agents, antimetabolites, nucleic acid, intercalating agents, topoisomerase inhibitors, agents which promote apoptosis, and/or agents which modulate immune responses. The effective amount of vitamin D₃ ester compound used can be modified according to the concentrations of the other agents used.

Changes in cell activity or cell proliferation can be used to determine whether the selected amounts are “effective amount” for the particular combination of compounds. The regimen of administration also can affect what constitutes an effective amount. As described in detail below, compounds of the invention can be administered to the subject prior to, simultaneously with, or after the administration of the other agent(s). Further, several divided dosages, as well as staggered dosages, can be administered daily or sequentially, or the dose can be proportionally increased or decreased as indicated by the exigencies of the therapeutic situation.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing compounds of the present invention include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these compositions include the step of bringing into association a compound of the invention with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound(s) of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compound(s) of the invention may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound(s) of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound of the invention include powders, sprays, ointments, pastes, creams, lotions, gels, solutions, patches and inhalants. The active compound(s) of the invention may be mixed under sterile conditions with a pharmaceutically-acceptable carrier, and with any preservatives, buffers, or propellants which may be required.

The ointments, pastes, creams and gels may contain, in addition to compound(s) of the invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to compound(s) of the invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound(s) of the invention can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compound(s) of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound(s) of the invention in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound(s) of the present invention are administered as pharmaceuticals, to humans and/or animals, they can be given per se or as a pharmaceutical composition containing, for example, 0.1 to 99.5% (more preferably, 0.5 to 90%) of active ingredient in combination with a pharmaceutically acceptable carrier.

These compound(s) may be administered to a “subject,” e.g., mammals, e.g., humans and other animals. Administration can be carried out by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compound(s) of the invention, which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient. Exemplary dose range is from 0.1 to 10 mg per day.

### Uses of the Compounds of the Invention

Another aspect of the invention pertains to compounds of the invention having at least one biological activity of vitamin D₃, and having improved biological properties when administered into a subject than vitamin D₃ under the same conditions, as well as, methods of testing and using these compounds to treat disorders involving an aberrant activity of hyperproliferative skin cells, parathyroid cells and bone cells.

Exemplary systems and assays for testing non-geiomic activity are extensively described in the following references: liver (Baran D. T. et al. (1989) FEBS Lett 259:205-208 and Baran D. T. et al. (1990) J. Bone Miner Res. 5:517-524; rat osteoblasts, e.g., ROS 17/2.8 cells (Baran D. T. et al. (1991) J. Bone Miner Res. 6:1269-1275, Caffrey J. M. (1989) J. Biol. Chem. 264:20265-20274 and Civitelli R. et al. (1990) Endocrinology 127:2253-2262); muscle (DeBoland A. R. and Boland R. L. (1993) Biochem. Biophys Acta Mol. Cell Res. 1179:93-104, Morelli S. et al. (1993) Biochem J. 289:675-679 and Selles J. and Boland R. L. (1991) Mol. Cell Endocrinol. 82:229-235); and in parathyroid cells (Bourdeau A. et al. (1990) Endocrinology 127:2738-2743).

Following binding, the transcriptional activity of a target gene (i.e., a gene associated with the specific DNA sequence) is enhanced as a function of the ligand bound to the receptor heterodimer. Exemplary vitamin D₃-responsive genes include osteocalcin, osteopontin, calbindins, parathyroid hormone (PTH), 24-hydroxylase, and α_{V}β₃-integrin. Genomic activities elicited by compounds of the invention can be tested by detecting the transcriptional upregulation of a vitamin D₃ responsive gene in a cell containing VD₃R_{S}. For example, the steady state levels of responsive gene mRNA or protein, e.g. calbindin gene, osteocalcin gene, can be detected in vivo or in vitro. Suitable cells that can be used include any vitamin D₃ responsive cell, e.g., keratinocytes, parathyroid cells, MG-63 cell line, ROS-17/2.8, among others.

In accordance with a still further embodiment of the present invention, convenient screening methods can be established in cell lines containing VD₃R_{S}, comprising (i) establishing a culture of these cells which include a reporter gene construct having a reporter gene which is expressed in an VD₃R-dependent fashion; (ii) contacting these cells with compounds of the invention; and (iii) monitoring the amount of expression of the reporter gene. Expression of the reporter gene reflects transcriptional activity of the VD₃R_{S} protein. Typically, the reporter gene construct will include a reporter gene in operative linkage with one or more transcriptional regulatory elements responsive to VD₃R_{S}, e.g. , the VD₃R_{S} response element (VDRE) known in the art. The amount of transcription from the reporter gene may be measured using any method known to those of skill in the art to be suitable. For example, specific mRNA expression may be detected using Northern blots or specific protein product may be identified by a characteristic stain, immunoassay or an intrinsic activity. In preferred embodiments, the gene product of the reporter is detected by an intrinsic activity associated with that product. For instance, the reporter gene may encode a gene product that, by enzymatic activity, gives rise to a detection signal based on color, fluorescence, or luminescence. The amount of expression from the reporter gene is then compared to the amount of expression in either the same cell in the absence of the test compound or it may be compared with the amount of transcription in a substantially identical cell that lacks the specific receptors. Agonistic vitamin D₃ compounds can then be readily detected by the increased activity or concentration of these reporter genes relative to untransfected controls.

After identifying certain test compounds as potential agonists or antagonists of vitamin D₃ compounds, the practitioner of the subject assay will continue to test the efficacy and specificity of the selected compounds both in vitro and in vivo. Whether for subsequent in vivo testing, or for administration to an animal as an approved drug, agents identified in the subject assay can be formulated in pharmaceutical preparations, such as described above, for in vivo administration to an animal, preferably a human.

As described herein, the compounds of the present invention show improved biological properties as compared to their isomeric counterparts. The improved biological property may occur in both a tissue-specific and non-specific manner. For example, certain tissues may be capable of metabolizing esters of vitamin D₃ into unique metabolites that enhance in a tissue-specific manner the biological activities of this compound.

Compounds of the invention exhibit sustained release activity, which allows for reduced toxicity and increased efficiency and therapeutic effect. As shown in Example IV, the compounds of the invention exist primarily intracellularly, whereas the parent compounds exist primarily extracellularly. These data indicate that compounds of the invention are capable of releasing the parent compound over a prolonged period of time. In particular, the data show that 20-epimer compounds will have increased sustained release activity over the parent compounds. In addition, esters of vitamin D₃ are more stable in vivo than vitamin D₃ itself. Any compound of the invention that shows significantly higher concentrations after prolonged incubations in vivo or in vitro, or that shows an increase in the binding to plasma vitamin D binding protein (DBP) compared to its isomeric counterpart is classified as a compound having enhanced stability (See A. W. Norman et al. J. Biol. Chem. 268 (27):20022-20030).

In the past, vitamin D₃ analogs have had limited clinical application due to hypercalcemia or deregulation of calcium homeostasis. However, the present invention provides compounds that, while retaining vitamin D₃ biological activities, have reduced hypercalcemic activity. Preferred compounds of the invention exhibit reduced calcium mobilization activity in vivo as exemplified by a marked decrease in intestinal calcium transport (ICA) and bone calcium mobilization (BCM) when compared to their non-epimeric counterparts. Thus, the dissociation of the biological activities (cell differentiation, immune effects) from the reduced deregulatory effect on calcium homeostasis provides vitamin D₃ ester compounds of the invention having significant therapeutic advantages over the parent compounds.

Compounds exhibiting reduced hypercalcemic activity can be tested in vivo or in vitro using methods known in the art and reviewed by Boullion, R. et al. (1995) Endocrinology Reviews 16(2):200-257. For example, the serum calcium levels following administration of a vitamin D₃ compound can be tested by routine experimentation (Lemire, J. M. (1994) Endocrinology 135(6):2818-2821). Briefly, compounds of the present invention can be administered intramuscularly to vitamin D₃-deficient subjects, e.g., rodents, e.g mouse, or avian species, e.g. chick. At appropriate time intervals, serum calcium levels and extent of calcium uptake can be used to determine the level of bone calcium mobilization (BCM) and intestinal calcium absorption (ICA) induced by the tested vitamin D₃ compound described in Norman, A. W. et al. (1993) J. Biol. Chem. 268(27):20022-20029. Compounds which upon addition fail to increase the concentration of calcium in the blood serum, thus showing decreased BCM and ICA responses compared to their isomeric counterparts, are considered to have reduced hypercalcemic activity. Compounds which have reduced toxicity compared to their isomeric counterparts are considered to have reduced toxicity. Additional calcium homeostasis-related assays are described below in the Calcium and Phosphate Homeostasis section.

### Hyperproliferative Conditions

In another aspect the present invention provides a method of treating in a subject, a disorder characterized by aberrant activity of a vitamin D₃-responsive cell. The method involves administering to the subject an effective amount of a pharmaceutical composition of a compound of the invention such that the activity of the cell is modulated.

In accordance with the present invention, compounds of the invention can be used in the treatment of both pathologic and non-pathologic proliferative conditions characterized by unwanted growth of hyperproliferative skin cells. In other embodiments, the cells to be treated are aberrant secretory cells, e.g. , parathyroid cells.

The use of vitamin D₃ compounds in treating hyperproliferative conditions has been limited because of their hypercalcemic effects. The present invention provides highly potent inhibitors of keratinocyte proliferation, which show reduced hypercalcemic activity compared to their isomeric counterparts. Thus, compounds of the invention provide a less toxic alternative to current methods of treatment.

In one embodiment, this invention features a method for inhibiting the proliferation and/or inducing the differentiation of a hyperproliferative skin cell, e.g., an epidermal or an epithelial cell, e.g. a keratinocytes, by contacting the cells with a compound of the invention. In general, the method includes a step of contacting a pathological or non-pathological hyperproliferative cell with an effective amount of compound of the invention to promote the differentiation of the hyperproliferative cells. The present method can be performed on cells in culture, e.g in vitro or ex vivo, or can be performed on cells present in an animal subject, e.g., as part of an in vivo therapeutic protocol. The therapeutic regimen can be carried out on a human or any other animal subject.

The compounds of the present invention can be used to treat a hyperproliferative skin disorder. Examples of these disorders include psoriasis, such as eczema; lupus associated skin lesions; psoriatic arthritis; rheumatoid arthritis that involves hyperproliferation and inflammation of epithelial-related cells lining the joint capsule; basal cell carcinoma; keratinization; dermatitides such as seborrheic dermatitis and solar dermatitis; keratosis such as seborrheic keratosis, senile keratosis, actinic keratosis. photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis.

As described above, compounds of the invention can be used to inhibit the hyperproliferation of keratinocytes in treating diseases such as psoriasis by administering an effective amount of these compounds to a subject in need of treatment. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis.

Pharmaceutical compositions of compounds of the invention can be delivered or administered topically or by transdermal patches for treating dermal psoriasis. Alternatively, oral administration is used. Additionally, the compositions can be delivered parenterally, especially for treatment of arthritis, such as psoriatic arthritis, and for direct injection of skin lesions. Parenteral therapy is typically intra-dermal, intra-articular, intramuscular or intravenous. A preferred way to practice the invention is to apply the vitamin D₃ compound, in a cream or oil based carrier, directly to the psoriatic lesions. Typically, the concentration of vitamin D₃ compound in a cream or oil is 1-2%. Alternatively, an aerosol can be used topically. These compounds can also be orally administered.

In general, the route of administration is topical (including administration to the eye, scalp, and mucous membranes), oral, or parenteral. Topical administration is preferred in treatment of skin lesions, including lesions of the scalp, lesions of the cornea (keratitis), and lesions of mucous membranes where such direct application is practical. Shampoo formulations are sometimes advantageous for treating scalp lesions such as seborrheic dermatitis and psoriasis of the scalp. Mouthwash and oral paste formulations can be advantageous for mucous membrane lesions, such as oral lesions and leukoplakia. Oral administration is a preferred alternative for treatment of skin lesions and other lesions discussed above where direct topical application is not as practical, and it is a preferred route for other applications.

Intra-articular injection is a preferred alternative in the case of treating one or only a few (such as 2-6) joints. Additionally, the therapeutic compounds are injected directly into lesions (intra-lesion administration) in appropriate cases. Intra-dermal administration is an alternative for dermal lesions such as those of psoriasis.

The amount of the pharmaceutical composition to be administered varies depending upon the type of the disease of a patient, the severity of the disease, the type of compound, among others. For example, a compound of the invention can be administered topically for treating hyperproliferative skin conditions at a dose in the range of 1 to 1000 mg per gram of topical formulation.

### Hormone Secretion

In yet another aspect, the present invention provides a method for modulating hormone secretion of a vitamin D₃ responsive cell, e.g., an endocrine cell, e.g., a parathyroid cell. Exemplary endocrine cells include parathyroid cells, among others.

The present method can be performed on cells in culture, e.g. in vitro or ex vivo, or on cells present in an animal subject, e.g., in vivo. Compounds of the invention can be initially tested in vitro, for example, by testing the inhibition of PTH secretion in response to compounds of the invention in parathyroid cells in culture. Other systems that can be used include the testing by prolactin secretion in rat pituitary tumor cells, e.g., GH4C1 cell line (Wark J. D. and Tashjian Jr. A. H. (1982) Endocrinology 111:1755-1757; Wark J. D. and Tashjian Jr. A. H. (1983) J. Biol. Chem. 258:2118-2121; Wark J. D. and Gurtler V. (1986) Biochem. J. 233:513-518) and TRH secretion in GH4C1 cells. Alternatively, the effects of compounds of the invention can be characterized in vivo using animals models as described in Nko M. et al. (1982) Miner Electrolyte Metab. 5:67-75; Oberg F. et al. (1993) J. Immunol. 150:3487-3495; Bar-Shavit Z. et al. (1986) Endocrinology 118:679-686; Testa U. et al. (1993) J. Immunol. 150:2418-2430; Nakamaki T. et al. (1992) Anticancer Res. 12:1331-1337; Weinberg J. B. and Larrick J. W. (1987) Blood 70:994-1002; Chambaut-Guérin A. M. and Thomopoulos P. (1991) Eur. Cytokine New. 2:355; Yoshida M. et al. (1992) Anticancer Res. 12:1947-1952; Momparler R. L. et al. (1993) Leukemia 7:17-20; Eisman J. A. (1994) Kanis JA (eds) Bone and Mineral Research 2:45-76; Veyron P. et al. (1993) Transplant Immunol. 1:72-76; Gross Metal. (1986) J. Bone Miner Res. 1:457-467; Costa E. M. et al. (1985) Endocrinology 117:2203-2210; Koga M. et al. (1988) Cancer Res. 48:2734-2739; Franceschi R. T. et al. (1994) J. Cell Physiol. 123:401-409; Cross H. S. et al. (1993) Naunyn Schmiedebergs Arch. Pharmacol. 347:105-110; Zhao X. and Feldman D. (1993) Endocrinology 132:1808-1814; Skowronski R. J. et al. (1993) Endocrinology 132:1952-1960; Henry H. L. and Norman A. W. (1975) Biochem. Biophys. Res. Commun. 62:781-788; Wecksler W. R. et al. (1980) Arch. Biochem. Biophys. 201:95-103; Brumbaugh P. F. et al. (1975) Am. J. Physiol. 238:384-388; Oldham S. B. et al. (1979) Endocrinology 104:248-254; Chertow B. S. et al. (1975) J. Clin Invest. 56:668-678; Canterbury J. M. et al. (1978) J. Clin. Invest. 61:1375-1383; Quesad J. M. et al. (1992) J. Clin. Endocrinol. Metab. 75:494-501.

In certain embodiments, compounds of the present invention can be used to inhibit parathyroid hormone (PTH) processing, e.g., transcriptional, translational processing, and/or secretion of a parathyroid cell as part of a therapeutic protocol. Therapeutic methods using these compounds can be readily applied to all diseases, involving direct or indirect effects of PTH activity, e.g., primary or secondary responses. For example, it is known in the art that PTH induces the formation of 1,25-dihydroxy vitamin D₃ in the kidneys, which in turn in increases calcium and phosphate absorption from the intestine that causes hypercalcemia. Thus inhibition of PTH processing and/or secretion would indirectly inhibit all of the responses mediated by PTH in vivo. Accordingly, therapeutic applications for these vitamin D₃ compounds include treating diseases such as secondary hyperparathyroidism of chronic renal failure (Slatopolsky E. et al (1990) Kidney Int. 38:S41-S47;Brown A. J. et al. (1989) J. Clin. Invest. 84:728-732). Determination of therapeutically affective amounts and dose regimen can be performed by the skilled artisan using the data described in the art.

### Calcium and Phosphate Homeostasis

The present invention also relates to a method of treating in a subject a disorder characterized by deregulation of calcium metabolism. This method comprises contacting a pathological or non-pathological vitamin D₃ responsive cell with an effective amount of a compound of the invention to thereby directly or indirectly modulate calcium and phosphate homeostasis. Techniques for detecting calcium fluctuation in vivo or in vitro are known in the art.

Exemplary Ca⁺⁺ homeostasis related assays include assays that focus on the intestine where intestinal ⁴⁵Ca²⁺ absorption is determined either 1) in vivo (Hibberd K. A. and Norman A. W. (1969) Biochem. Pharmacol. 18:2347-2355; Hurwitz S. et al. (1967) J. Nutr. 91:319-323; Bickle D. D. et al. (1984) Endocrinology 114:260-267), or 2) in vitro with everted duodenal sacs (Schachter D. et al. (1961) Am. J. Physiol 200:1263-1271), or 3) on the genomic induction of calbindin-D₂₈ₖ in the chick or of calbindin-D₉ₖ in the rat (Thomasset M. et al. (1981) FEBS Lett. 127:13-16; Brehier A. and Thomasset M. (1990) Endocrinology 127:580-587). The bone-oriented assays include:1) assessment of bone resorption as determined via the release of Ca²⁺ from bone in vivo (in animals fed a zero Ca²⁺ diet) (Hibberd K. A. and Norman A. W. (1969) Biochem. Pharmacol. 18:2347-2355; Hurwitz S. et al. (1967) J. Nutr. 91:319-323), or from bone explants in vitro (Bouillon R. et al. (1992) J. Biol. Chem. 267:3044-3051), 2) measurement of serum osteocalcin levels [osteocalcin is an osteoblast-specific protein that after its synthesis is largely incorporated into the bone matrix, but partially released into the circulation (or tissue culture medium) and thus represents a good marker of bone formation or turnover] (Bouillon R. et al. (1992) Clin. Chem. 38:2055-2060), or 3) bone ash content (Norman A. W. and Wong R. G. (1972) J. Nutr. 102:1709-1718). Only one kidney-oriented assay has been employed. In this assay, urinary Ca²⁺ excretion is determined (Hartenbower D. L. et al. (1977) Walter de Gruyter, Berlin pp 587-589); this assay is dependent upon elevations in the serum Ca²⁺ level and may reflect bone Ca²⁺ mobilizing activity more than renal effects. Finally, there is a “soft tissue calcification” assay that has been employed to detect the consequences of 1α,25(OH)₂D₃ or analog-induced severe hypercalcemia. In this assay a rat is administered an intraperitoneal dose of ⁴⁵Ca²⁺ followed by seven daily relative high doses of 1α,25(OH)₂D₃ or the analog of interest; in the event of onset of a severe hypercalcemia, soft tissue calcification can be assessed by determination of the ⁴⁵Ca²⁺ level. In all these assays, either compounds of the inventions or related analogs are administered to vitamin D-sufficient or vitamin D-deficient animals, as a single dose or chronically (depending upon the assay protocol), at an appropriate time interval before the end point of the assay is quantified.

In certain embodiments, compounds of the invention can be used to modulate bone metabolism. It is known in the art, that vitamin D₃ compounds exert effects on the bone forming cells, the osteoblasts through genomic and non-genomic pathways (Walters M. R. et al. (1982) J. Biol. Chem. 257:7481-7484; Jurutka P. W. et al.(1993) Biochemistry 32:8184-8192; Mellon W. S. and DeLuca H. F. (1980) J. Biol. Chem. 255:4081-4086). Similarly, vitamin D₃ compounds are known in the art to support different activities of bone resorbing osteoclasts such as the stimulation of differentiation of monocytes and mononuclear phagocytes into osteoclasts (Abe E. et al. (1988) J. Bone Miner Res. 3:635-645; Takahashi N. et al. (1988) Endocrinology 123:1504-1510; Udagawa N. et al. (1990) Proc. Natl. Acad. Sci. USA 87:7260-7264). Accordingly, compounds of the present invention that modulate the production of bone cells can influence bone formation and degeneration.

The present invention provides a method for modulating bone cell metabolism by contacting a pathological or a non-pathological bone cell with an effective amount of a compound of the invention to thereby modulate bone formation and degeneration. The present method can be performed on cells in culture, e.g., in vitro or ex vivo, or can be performed in cells present in an animal subject, e.g., cells in vivo. Exemplary culture systems that can be used include osteoblast cell lines, e.g., ROS 17/2.8 cell line, monocytes, bone marrow culture system (Suda T. et al. (1990) Med. Res. Rev. 7:333-366; Suda T. et al. (1992) J. Cell Biochem. 49:53-58) among others. Selected compounds can be further tested in vivo, for example, animal models of osteopetrosis and in human disease (Shapira F. (1993) Clin. Orthop. 294:34-44).

In a preferred embodiment, a method for treating osteoporosis is provided, comprising administering to a subject a pharmaceutical preparation of a vitamin D₃ compound to thereby ameliorate the condition relative to an untreated subject. The rationale for utilizing vitamin D₃ compounds in the treatment of osteoporosis is supported by studies indicating a decrease in serum concentration of 1α,25(OH)₂D₃ in elderly subjects (Lidor C. et al. (1993) Calcif. Tissue Int. 52:146-148). In vivo studies using vitamin D₃ compounds in animal models and humans are described in Bouillon, et al. (1995) Endocrine Reviews 16(2):229-23 1.

Compounds of the invention can be tested in ovarectomized animals, e.g. , dogs, rodents, to assess the changes in bone mass and bone formation rates in both normal and estrogen-deficient animals. Clinical trials can be conducted in humans by attending clinicians to determine therapeutically effective amounts of the ester compounds in preventing and treating osteoporosis.

The compounds of the invention are useful in the treatment of senile osteoporosis. These compounds may be useful in treating osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hyperparathyroidism, cirrhosis, obstructive jaundice, drug induced metabolism, medullary carcinoma, chronic renal disease, hypophosphatemic VDRR, vitamin D-dependent rickets, sarcoidosis, glucocorticoid antagonism, malabsorption syndrome, steatorrhea, tropical sprue, idiopathic hypercalcemia and milk fever.

It is understood by the ordinarily skilled artisan that metabolism of a vitamin D₃ substrate into a 3-epi vitamin D₃ compound in a cell is indicative that such compound is biologically active in such cell, and thus that it can be used in treating conditions arising from aberrant activity of such cells. For example, production of 3-epi vitamin D₃ compounds in keratinocytes, smooth muscle cells and bone cells is indicative that such 3-epi vitamin D₃ compounds are biologically active in those cells and can be used in treating conditions such as psoriasis, hypertension and osteoporosis, respectively.

The invention is further illustrated by the following examples which in no way should be construed as being further limiting.

### EXAMPLES

### Example I

### Metabolism of 1α,25(OH)₂-16-ene-D₃ Analogs in Bone Cells

As described herein, various analogs of 1α,25(OH)₂-D₃, such as 1α,25(OH)₂-16-ene-D₃ analogs are metabolized into less polar metabolites in the rat osteosarcoma cell line UMR 106. UMR 106 cells were cultured in an humidified atmosphere at 37° C. in 95% air and 5% CO₂. MacCoy's culture medium, containing 10% fetal calf serum (FCS), antibiotics (100 IU/mL penicillin and 100 μg/mL streptomycin) and 22% calcium bicarbonate, was used. Cells became confluent ten days after seeding, and were then incubated with analog. Samples (structures shown in Table 2) of 1α,25(OH)₂-16-ene-D₃, 1α,25(OH)₂-16-ene-3-epi-D₃, 1α,25(OH)₂-16-ene-20-epi-D₃, 1α,25(OH)₂-16-ene-20-epi-3-epi-D₃, and 1α,25(OH)₂-16-ene-23-yne-D₃ were dissolved in ethanol to a final concentration of 10 μM and incubated in 50 mL for 24 h. Each analog was incubated in three culture bottles. Incubation was stopped by adding 10 mL methanol to each culture bottle. Culture bottles were stored at −20° C.

TABLE 2

| Compound Name | Structure |
|---|---|
| 1∝,25(OH)₂-16-ene-D₃ | 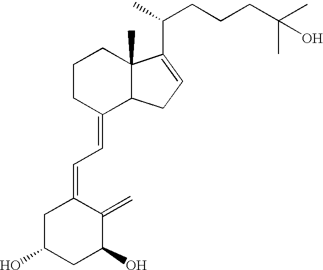 |
| 1∝,25(OH)₂-16-ene-3-epi-D₃ | 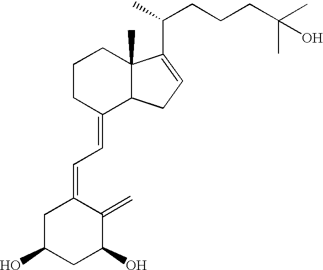 |
| 1∝,25(OH)₂-16-ene-20-epi-D₃ | 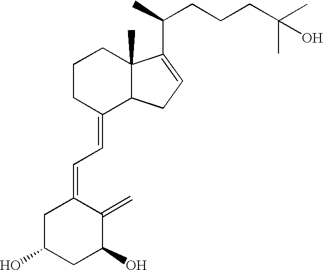 |
| 1∝,25(OH)₂-16-ene-20-epi-3-epi-D₃ | 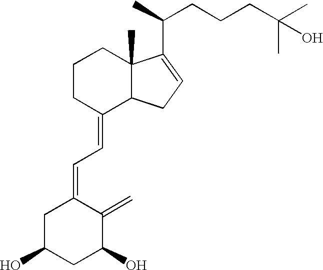 |
| 1∝,25(OH)₂-16-ene-23-yne-D₃ | 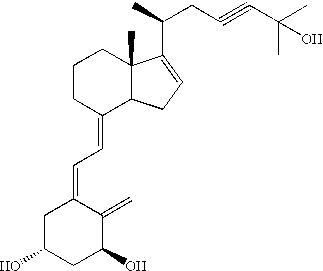 |

### Example II

### Isolation of Metabolites of 1α,25(OH)₂-16-ene-D₃ Analogs

Lipid extraction was initiated by first adding two volumes of methanol to each culture bottle from Example I. The protein precipitate was separated from the supernatant by centrifugation at 3000 rpm at 4° C. for 15 min. The supernatant was mixed with four volumes of dichloromethane in a separatory funnel. The lower organic phase was collected and dried under nitrogen gas at 50° C. After reconstitution in 10% isopropanol/hexane, the lipid extract was analyzed by high-performance liquid chromatography (HPLC).

HPLC was performed with a Waters System Controller (Model 600E) equipped with a photodiode array detector (Model PDA 990) to monitor UV absorption at 265 nm. A Zorbax SIL 9.4×250 mm column (DuPont, Wilmington, Del.) was used for all straight phase systems. The corresponding analog was added to each lipid extract and the solutions were then subjected to a straight phase HPLC system using 10% isopropanol/hexane at a flow rate of 2 mL/min. (HPLC system I). Fractions were collected from 0 min. to 12 min. These fractions were further subjected to a straight phase HPLC system using 2% isopropanol/hexane at a flow rate of 2 mL/min. (HPLC system II).

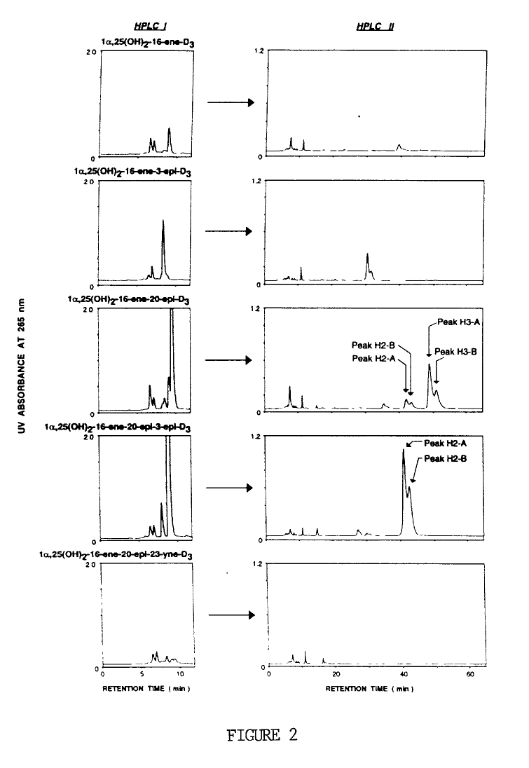

As shown in FIG. 1, HPLC system I analysis revealed less polar peaks (in the 5-10 minute region of the chromatogram) for each metabolite. FIG. 2 shows the HPLC profile using HPLC system II. As indicated in FIG. 2, the less polar peaks of 1α,25(OH)₂-16-ene-20-epi-D₃ and 1α,25(OH)₂-16-ene-20-epi-3-epi-D₃ analoga were referred to as H2-A, H2-B, H3-A and H3-B.

### Example III

### Identification of Metabolites of 1α,25(OH ₂-16-ene-D₃ Analogs

UMR 106 cells were cultured as described in Example 1. After confluent, cells were incubated with 10 μM of 1α,25(OH)₂-16-ene-20-epi-D₃ or 1α,25(OH)₂-16-ene-20-epi-3-epi-D₃ in 50 mL of medium for 24 hr. Incubation was stopped by adding 10 mL of methanol to each culture bottle. Culture bottles were stored at −20° C.

Lipid extraction was carried out as described in Example II. HPLC was performed with a Waters System Controller (Model 600E) equipped with a photodiode array detector (Model PDA 990) to monitor UV absorption at 265 nm. A Zorbax SIL 9.4×250 mm column (DuPont, Wilmington, Del.) was used for all straight phase systems and a Zorbax ODS 4.6×250 nm column (DuPont, Wilmington, Del.) was used for all reverse phase systems. All HPLC analysis was performed at a flow rate of 2 mL/min. Table 3 summarizes the HPLC results.

TABLE 3
Isolation and identification of peaks H2-A, H2-B, H3-A, and H3-B
Isolation of peaks H2 and H3

| | Mobile Phase | Peak H2 | | Peak H3 | |
|---|---|---|---|---|---|
| HPLC I | 10% | 0-12 | min. | 0-12 | min. |
| Straight Phase | isopropanol/hexane | | | | |
| HPLC II | 10% | 35-50 | min. | 44-56 | min. |
| Straight Phase | isopropanol/hexane | | | | |

### Example IV

### Distribution and Metabolism of Metabolites of 1α,25(OH)₂-16-ene-20-epi-D₃ Analogs in Bone Cells

Peaks H2-A and H3-A were obtained and purified as described in Example II. In addition, UMR 106 cells were incubated with 1α,25(OH)₂-16-ene-20-epi-D₃ and 1α,25(OH)₂-16-ene-20-epi-3-epi-D₃, as described in Example I.

The lipid extraction was carried out from media and cells separately and together, as described in Example II. HPLC was performed with a Waters System Controller (Model 600E) equipped with a photodiode array detector (Model PDA 990) to monitor UV absorption at 265 nm. A Zorbax SIL 9.4×250 mm column (DuPont, Wilmington, Del.) was used for all straight phase systems. Each lipid extract was analyzed using HPLC system I.

The esters of 1α,25(OH)₂-16-ene-20-epi-D₃ and 1α,25(OH)₂-16-ene-20-3-epi-D₃ were found to be mostly in the UMR 106 cells. The substrates, i.e. 1α,25(OH)₂-16-ene-20-epi-D₃ or 1α,25(OH)₂-16-ene-20-epi-3-epi-D₃, were distributed primarily in the media.

The esters of 1α,25(OH)₂-16-ene-20-epi-D₃ and 1α,25(OH)₂-16-ene-20-epi -3-epi-D₃ were also found mostly in the Ros 17/2.8 cells. Again, the substrates of these compounds were found distributed in the media. UV spectra of the substrate compounds found in several of the HPLC fractions from both UMR 106 and Ros 17/2.8 cells were also compared.

### Example V

### NMR Analysis of Metabolites of 1,25-Dihydroxy-16-ene-20-epi-D₃ and Metabolites of 1,25-Dihydroxy-16-ene-20-epi-3-epi-D₃

Two metabolites (3A and 3B) of 1,25-Dihydroxy-16-ene-20-epi-D₃ (Ro 25-8845) were examined by HNMR spectroscopy. The NMR spectra of deuterochloroform solutions of these metabolites were compared with the spectrum of the parent compound, also dissolved in deuterochloroform, in order to determine the structural differences between the metabolites and the parent compound. The most significant difference for both metabolites is the shift of H-1 from 4.45 ppm in the spectrum of the parent compound to 5.51 ppm in the spectra of the metabolites, with no significant change in coupling constants. A modification of the geometry of ring A is ruled out based on decoupling experiments and the similarity with the parent compound spectrum. Keeping ring A intact and shifting H-11.06 ppm downfield can best be explained by the acetylation effect.

Both 3A and 3B show a 2-proton triplet at 2.26 ppm indicating the presence of a methylene group attached to a carbonyl which is a characteristic feature of an ester of a fatty acid 3B, in addition, shows the presence of a 2-proton alkene triplet at 5.34 ppm and a 4-proton band at 2.01 ppm indicating the presence of a double blond flanked by at least a 2-methylene chain on each side. This is characteristic of an ester of a monounsaturated fatty acid.

The NMR data is consistent with each metabolite being an ester of a fatty acid with esterification occurring at C-1. 3A is assigned as a RO 25-8845 ester of a saturated fatty acid and 3B is assigned as a RO 25-8845 ester of a monounsaturated fatty acid. Evidence for the methylene envelope and a terminal methyl group of a fatty ester chain is obscured by the presence of a large hexane impurity in the spectra of both metabolites.

Two metabolites (2A and 2B) of 1,25-Dihydroxy-16-ene-3-epi-20-epiD₃ (RO 27-3509) were similarly analyzed by HNMR. The NMR spectra of deuterochloroform solutions of these two metabolites were compared with the spectrum of the parent compound, also dissolved in deuterochloroform. Comparing both the HNMR spectrum of the parent compound. Identical results were obtained. The H-1 proton is shifted to 5.37 ppm in the metabolite spectra from 4.31 ppm in the spectrum of the parent compound. Both metabolite spectra show a 2-proton methylene triplet at 2.31 ppm. 2B shows a 2-proton alkene triplet at 5.34 ppm and a 4-proton methylene band at 2.01 ppm. The spectra of 2A and 2B also show a large Hexane impurity. The NMR data for these metabolites are consistent with 2A and 2B being esters of fatty acids with esterification occurring at C-1. 2A is assigned as an RO 27-3509 ester of a saturated fatty acid and 2B is assigned as an RO 27-3509 ester of a monounsaturated fatty acid.

### Example VI

### Mass Spectrometric Characterization of Metabolites of 1α,25(OH)₂-16-ene-D₃ Analogs

The compounds represented by peaks H2 and H3 (see FIG. 2 ) were identified by gas chromatography-mass spectrometry (GC-MS). Briefly summarizing, analysis of the GC-MS data in combination with the results of the HNMR study and analysis described in Example V above indicated that: compound H2-A (compound 2A of Example V) is 3-epi-25-hydroxy-16-ene-20-epi-D₃-1-α-stearate; H2-B (compound 2B of Example V) is 3-epi-25-hydroxy-16-ene-20-epi-D₃-1-α-oleate; H3-A (compound 3A) is 25-hydroxy-16-ene-20-epi-D₃-1-α-stearate; and H3-B (compound 3B) is 3-epi-25-hydroxy-16-ene-20-epi-D₃-1-α-oleate.

### Procedure for GC-MS Study

Trimethylsilylated 1α,25-dihydroxy-16-ene-20-epi-D3 was used as a standardin a gas chromatogram and electron impact mass spectrum. The major peak at 23.18 minutes yields mass spectral characteristics typical of vitamin D-TMS derivatives: a weak molecular ion at m/z 630, sequential losses of trimethylsilanol at m/z 540 and 450, cleavage of the C24-C25 bond at m/z 131, confirmation of C1 and C3 hydroxylation at m/z 217, and the diagnostic loss of 131 Da from the A-ring, yielding a product ion at m/z 499.

ESI-ITMS of underivatized H2-A yielded an intense [M+Na]⁺ ion at m/z 703.5 which, upon collision in MS², produced a prominent fragmentation product at m/z 419.2. The large shift in mass from the parent compound implies the presence of a rather large modification to the structure, and the intensity with which the m/z 419.2 fragment is produced is unusual for most underivatized vitamin D metabolites. The MS data acquired for the underivatized H2-A was then supplemented by analysis of its corresponding PTAD derivative. PTAD, which targets the cisoid diene region of the vitamin D seco-steroid molecule, offers both improved ionization characteristics and means to detect vitamin D metabolites by their mass shift; PTAD derivatization adds 175 Da to the mass of vitamin D compounds, and comparison against underivatized spectra enables their rapid identification. The derivatization yielded an [M+Na]⁺ ion at m/z 878.3, as predicted. Collision of this ion in MS² produced major ion product at mTh/z 594.4, reflecting a 284 Da neutral loss identical to that of the underivatized H2-A material. Further analysis of the m/z 594.4 ion in MS³ revealed that both the vitamin D core and the PTAD tag remained intact within the fragment ion, implying that the neutral loss of 284 represented, in toto, the substituent added by metabolic processes.

GC-MS analysis of fraction H2-A produced a chromatogram which was screened for vitamin D-specific diagnostic ions at m/z 131 and 217. Individual peaks of the chromatogram were also examined for other vitamin D characteristics (consecutive losses of trimethylsilanol) and revealed that fraction H2-A yielded vitamin D compounds at 20.70, 21.99, 23.88, and 24.39 minutes. The mass spectra corresponding to these peaks contain important structural data. In all cases, the ions at m/z 540 are not accompanied by corresponding fragments 41 Da lower. This indicates that mere stereochemical alteration alone has not occurred, as this would have maintained a derivatized molecular weight of 630 Da. In the absence of evidence to the contrary, m/z 540 was believed to be the molecular ion of these species. The presence of the fragment ions at m/z 131 further indicates that metabolic modification did not take place at the hydroxyisopropyl group at the end of the sidechain.

Perhaps most significant is the omission of a number of fragment ions which were present in the spectrum of the standard. The m/z 217 fragment ion, usually a prominent fragment found in most 1-hydroxylated vitamin D compounds, is entirely absent from all of these spectra; disruption of this fragmentation pathway suggests a modification to the A-ring. Assuming that the molecular ion resides at m/z 540, A-ring modification would also conceivably interfere with the formation of a corresponding [M-131]⁺ fragment ion at m/z 409. A post-derivatization molecular weight of 540 Da is also rather reminiscent of the trimethylsilanol losses encountered with the standard upon fragmentation by electron impact. The mass spectral evidence implicating the A-ring as the site of metabolism, in addition to these apparent 90 Da mass shifts when compared to the substrate, provides compelling evidence of A-ring dehydration. Due to the symmetry about the A-ring, it cannot be established whether the site of the dehydration involved elimination of the C1 hydroxyl or the C3 hydroxyl group. The presence of a double bond at either of these two locations would also interfere with the loss of the C2-C4 fragment that would normally yield the [M-131 ]⁺ ion, and thus cannot be exploited to differentiate between the two possible structures.

From interpretation of the ESI-ITMS and GC-MS results, it appears that A-ring dehydration detected in GC-MS analysis of H2-A parallels that caused by collision-induced dissociation in the ion trap. The unsodiated, underivatized molecular weight of the vitamin D fragment produced in ESI-ITMS is 396 Da. Accounting for the presence of two hydroxyl groups on the molecule, one can then calculate a projected molecular weight for this fragment upon trimethylsilylation for GC-MS analysis. The addition of two trimethylsilyl groups, each contributing 72 Da tot he metabolite mass, results in a final mass of 540 Da: precisely the mass of the vitamin D species detected in the GC chromatogram for fraction H2-A.

With this relationship established, it was proposed that the neutral loss encountered upon fragmentation in the ion trap mass spectrometer was being liberated from the metabolite before or upon introduction of the analyte to the GC column, i.e., while the 284 Da neutral could be released from the analyte under the controlled conditions of an MS² experiment in an ion trap, this same 284 Da moiety was suspected of being eliminated prematurely from the metabolite structure due to thermal degradation in the injection port. Such an elimination conceivably would produce an unsaturation in its place on the A-ring, and thus the A-ring dehydration experienced in GC-MS was likely an artifact of the technique. This particularly holds true if the metabolite is an ester; the thermally-induced elimination of esters is a well-understood pyrolytic process, frequently used by synthetic chemists to produce olefinic bonds in high yield. Though the temperatures necessary for this reaction are dependent on the esters involved, 300° C. is often sufficient to cause this elimination to take place.

Even though the GC-MS is therefore suspected of propagating artifactual vitamin D analyte species from fraction H2-A, identification of the products of this degradative process could aid in the characterization of the metabolite. Because this proposed mechanism results in elimination of this 284 Da moiety in the injection port, it follows that this degradation product could be detected as its own non-vitamin D-related analyte.

The mass spectrum corresponding to the chromatographic peak at 14.45 minutes contains none of the typical vitamin D diagnostic ion fragments, but the molecular ion at m/z 356 is precisely 72 Da greater than 284 Da, and thus represents the neutral loss observed in the ion trap. Upon thermal elimination of this species in the injection port, this 284 Da moiety was immediately derivatized by residual vapors of the trimethylsilylation reagent; given that the GC inlet compartment is purged 2 minutes after injection, there is ample time for such a facile reaction to take place. Of additional significance is the base peak at m/z 117, which indicative of a trimethylsilylated carboxylic acid. The paucity of intense fragmentation in the mid-mass region of the spectrum (m/z 150-340) suggested that the remainder of the molecule was likely comprised of aliphatic hydrocarbon. The addition of methylene units to the m/z 117 fragment led to the conclusion that this moiety was the saturated C₁₈ fat, stearic acid. Especially strong confirmation was provided by the on-line mass spectrum library, which verified our conclusions with a confidence rating 96%. NMR data (see Example V) was used to further establish the site of stearic acid attachment at the C-1 carbon on the A-ring. Therefore, based on structural information derived collectively from GC-MS, ESI-ITMS, and NMR, the compound H2-A is 3-epi-25-hydroxy-16-ene-20-epi-D₃-1-stearate.

An analogous relationship between ESI-ITMS and GC-MS results was encountered with H-2B. Analysis of the underivatized metabolite produces an ion at m/z 701.3, which is shifted upwards to m/z 876.5 upon derivatization with PTAD. Fragmentation of the m/z 876.5 ion by MS² yields the m/z 594.3 fragment, which again represents the PTAD and the vitamin D portion of the metabolite; the neutral loss of 282 Da is precisely the molecular weight of a monounsaturated C₁₈ fatty acid.

The vitamin D analyte profile in the GC chromatogram trimethylsilylated H2-B is essentially identical to that of fraction H2-A. Peaks found at 20.69, 21.99, 23.85, and 24.35 minutes all yield mass spectra indicative of A-ring dehydration and unaltered sidechains. Magnified detail of the peak at 14.28 minutes reveals a closely eluting series of four peaks, in which the last of the four exhibits the same retention time as trimethylsilylated stearic acid. The major peak in this region of the chromatogram, had a retention time 14.28 minutes. In a mass spectrum of this peak, an ion fragment at m/z 117 indicated that these analytes are also trimethylsilylated fatty acids, whose molecular ions at m/z 354 are entirely consistent with those of monounsaturated C₁₈ fatty acids.

Library searching of the mass spectra for these compounds cannot easily resolve which C₁₈:Δ1 fatty acid isomer is presented by each peak, but all results strongly confirm their identities as monounsaturated C₁₈ acids and therefore the metabolites as esters of these fatty acids. Given the prevalence of the 9-cis isomer in nature, it is likely that the most abundant metabolite infraction H2-B (whose lipid portion is represented by the chromatographic peak at 14.28 minutes) is 3-epi-25-hydroxy-16-ene-20-epi-D₃-1-oleate.

H3-A yielded ESI-ITMS data similar to that found with H2-A: the underivatized pseudomolecular ion at m/z 703.6 and post derivatization ion at m/z 878.5 both exhibit losses of 284 Da upon collision-induced dissociation. Analysis of H3-A as its trimethylsilyl derivative produced a somewhat different profile of vitamin D chromatographic peaks; at least five peaks between 20-25 minutes yielded mass spectra consistent with A-ring dehydrated species. The peak at 20.69 minutes, which yielded a putative molecular ion at m/z 450, could suggest possible bis-dehydration of the A-ring, but little other evidence supports this conclusion. The base peak in the chromatogram at 14.45 minutes exhibits the same retention time as that of the trimethylsilyl stearic acid component in H2-A. Furthermore, the mass spectrum of this peak is highly consistent with that of TMS-derivatized octadecanoic fatty acid, and a library match confirms the assignment with 91% confidence. Given that H3-A is attributable to the 3β substrate, it follows that this metabolite is the 3β analogue of H2-A, and is therefore assigned the structure 25-hydroxy-16-ene-20-epi-D₃-1-stearate.

Analogous to the comparisons made between H2-A and H2-B, fraction H3-B yields both ESI-ITMS data and a series of vitamin D-related GC degradation products similar to that of H3-A. However, close examination of the chromatogram at 14.28 minutes reveals a fatty acid profile practically identical to that seen with H2-B. Subsequent interpretation and library search of these spectra support the conclusion that H3-B is a mixture of octadecenoic acid conjugates of 1α,25(OH)₂-16-ene-20-epi-D₃. As was the case with fraction H2-B, the absence of suitable standards does not permit the definitive assignment of a specific C₁₈:Δ1 fatty acid isomer, but based on the abundance of oleate in nature, the major component of this isomeric mixture is identified as 25-hydroxy-16-ene-20-epi-D₃-1-oleate.

### Incorporation by Reference

All patents, published patent applications and other references disclosed herein are hereby expressly incorporated herein in their entireties by reference.

### Equivalents

Those skilled in the art will recognize, or be able to ascertain using no more than routine experimentation, many equivalents of the specific embodiments of the invention described herein. Such equivalents are intended to be encompassed by the following claims.

What is claimed is:

1. An isolated form of a vitamin D₃ compound having formula I: 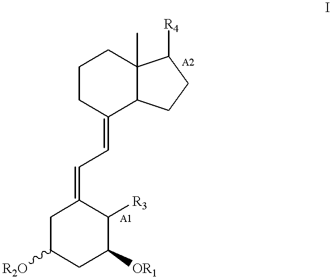
wherein:
A₁ is a single or double bond; A₂ is a single bond or a double bond;
R₁ and R₂ are each hydrogen or a hydrolyzable moiety, provided that R₁ and R₂ are not both hydrogen;
R₃ is hydrogen, deuterium, deuteroalkyl, hydroxyl, alkyl, alkoxide, O-acyl, halogen, haloalkyl, hydroxyalkyl, amino or thiol; and
R₄ is a saturated or unsaturated carbon chain represented by the formula: 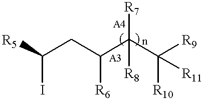
wherein I represents the above-described formula I;
A₃ and A₄ are each, independently, a single bond or a double bond;
R₅, R₆, R₇, and R₈, are each, independently, hydrogen, deuterium, hydroxyl, alkyl, alkoxide, O-acyl, halogen, haloalkyl, hydroxyalkyl, oxygen, amino or thiol;
R₉ and R₁₀ are each, independently, alkyl, hydroxyalkyl, halogen, hydroxyl, haloalkyl or deuteroalkyl;
R₁₁ is hydrogen, hydroxyl or O-acyl; and
n is an integer from 1 to 5.

2. The compound of claim 1 wherein A₁ is a double bond, A₂, A₃ and A₄ are single bonds, R₆, R₇ and R₈ are hydrogen, R₅, R₉ and R₁₀ are methyl, n is 1, and the substituent R₂O at the 3-carbon position is in the β-configuration.

3. The compound of claim 1 wherein R₂ is hydrogen.

4. The compound of claim 1 wherein A₁ is a double bond.

5. The compound of claim 1 wherein A₂ is a double bond.

6. The compound of claim 1 wherein R₃ is methyl.

7. The compound of claim 1 wherein R₅ is methyl.

8. The compound of claim 1 wherein R₁₁ is hydroxyl.

9. The compound of claim 1 wherein R₁₁ is hydrogen.

10. The compound of claim 1 wherein R₁ has the formula—C(═O)R₁₃, wherein R₁₃ is C₁-C₂₆alkyl, aryl or aralkyl.

11. The compound of claim 10 wherein R₁₃ has the formula —(CH₂)ₓ—CH═CH—(CH₂)_{y}—CH₃, wherein x and y are an integer from 1 to 10.

12. The compound of claim 10 wherein R₁₃ has the formula —(CH₂)₂CH₃, wherein z is an integer from 1 to 25.

13. The compound of claim 10 wherein R₁₃ is a side chain of a fatty acid.

14. The compound of claim 13 wherein R₁₃ is a side chain of a naturally occurring fatty acid.

15. The compound of claim 14 wherein the side chain is a side chain of lauric acid, myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, or cerebronic acid.

16. The compound of claim 10 wherein the R₂O substituent at the 3-carbon position is in the α-configuration

17. The compound of claim 10 wherein the R₂O substituent at the 3-carbon position is in the β-configuration.

18. The compound of claim 16 or 17 wherein R₂ is hydrogen.

19. An isolated form of a vitamin D₃ compound having formula II: 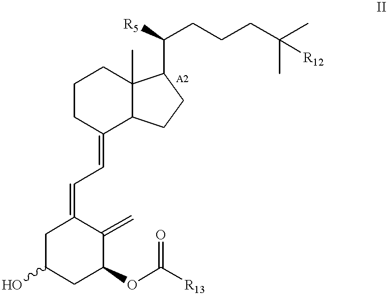
wherein:
A₂ is a single bond or a double bond;
R₅ is deuterium, hydroxyl, alkyl, alkoxide, O-acyl, halogen, haloalkyl, hydroxyalkyl, oxygen, amnino or thiol;
R₁₂ is hydrogen, hydroxyl or O-acyl; and
R₁₃ is C₁-C₂₆ alkyl, aryl or aralkyl.

20. The compound of claim 19 wherein A₂ is a single bond, the hydroxyl substituent at the 3-carbon position is in the β-configuration, and R₁₂ is hydrogen.

21. The compound of claim 19 wherein R₁₃ has the formula —(CH₂)ₓ—CH═CH—(CH₂)_{y}—CH₃, wherein x and y are an integer from 1 to 10.

22. The compound of claim 19 wherein R₁₃ has the formula —(CH₂)_{z}CH₃, wherein z is an integer from 1 to 25.

23. The compound of claim 19 wherein R₁₃ is a side chain of a fatty acid.

24. The compound of claim 23 wherein R₁₃ is a side chain of a naturally occurring fatty acid.

25. The compound of claim 24 wherein the side chain is a side chain of lauric acid, myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, or cerebronic acid.

26. The compound of claim 25 wherein the side chain is a side chain of stearic acid or oleic acid.

27. The compound of claim 19 wherein the hydroxyl group at the 3-carbon position is in the α-configuration.

28. The compound of claim 19 wherein the hydroxyl group at the 3-carbon position is in the β-configuration.

29. The compound of claim 19 wherein R₁₂ is hydroxyl.

30. The compound of claim 19 wherein R₁₂ is hydrogen.

31. An isolated form of a vitamin D₃ compound having formula III: 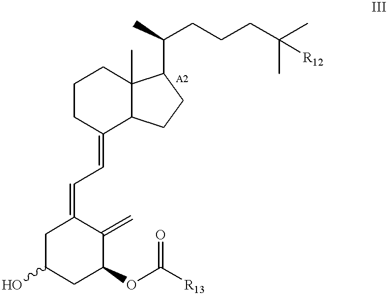
wherein:
A₂ is a single bond or a double bond;
R₁₂ is hydrogen or hydroxyl; and
R₁₃ is a side chain of a naturally occurring fatty acid.

32. The compound of claim 31 wherein the side chain is a side chain of lauric acid, myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, or cerebronic acid.

33. The compound of claim 31 wherein the hydroxyl group at the 3-carbon position is in the α-configuration.

34. The compound of claim 31 wherein the hydroxyl group at the 3-carbon position is in the β-configuration.

35. The compound of claims 33 or 34 wherein R₁₂ is hydroxyl.

36. The compound of claims 33 or 34 wherein R₁₂ is hydrogen.

37. The compound of claims 33 or 34 wherein the side chain is a side chain of stearic acid or oleic acid.

38. The compound of claim 31 which is 3-epi-25-hydroxy-16-ene-20-epi-D₃-1-α-stearate, 3-epi-25-hydroxy-16-ene-20-epi-D₃-1-α-oleate, 25-hydroxy-16-ene-20-epi-D₃-1-α-stearate, 25-hydroxy-16-ene-20-epi-D₃-1-α-oleate, 3-epi-25-hydroxy-20-epi-D₃-α-stearate, 3-epi-25-hydroxy-20-epi-D₃-1-α-oleate, 25-hydroxy-20-epi-D₃-1-α-stearate, or 25-hydroxy-20-epi-D₃-1-α-oleate.

39. A method of treating a disorder characterized by an aberrant activity of a vitamin D₃-responsive cell, comprising administering to a subject an effective amount of a compound of claim 1, such that the aberrant activity of the vitamin D₃-responsive cell is reduced.

40. The method of claim 39 wherein the compound has at least one improved biological property compared to vitamin D₃ under the same conditions.

41. The method of claim 40 wherein the at least one improved biological property comprises a reduction in hypercalcemia compared to the hypercalcemia induced by vitamin D₃ under the same conditions.

42. The method of claim 40 wherein the at least one improved biological property comprises an enhanced stability of the compound compared to vitamin D₃ under the same conditions.

43. The method of claim 39 wherein the disorder comprises an aberrant activity of a hyperproliferative skin cell.

44. The method of claim 43 wherein the disorder is selected from psoriasis, basal cell carcinoma and keratosis.

45. The method of claim 39 wherein the disorder comprises an aberrant activity of an endocrine cell.

46. The method of claim 45 wherein the endocrine cell is a parathyroid cell and the aberrant activity is processing and/or secretion of parathyroid hormone.

47. The method of claim 46 wherein the disorder is secondary hyperparathyroidism.

48. The method of claim 39 wherein the disorder comprises an aberrant activity of a bone cell.

49. The method of claim 48 wherein the disorder is selected from osteoporosis, osteodystrophy, senile osteoporosis, osteomalacia, rickets, osteitis fibrosa cystica, and renal osteodystrophy.

50. The method of claim 39 wherein the disorder is cirrhosis or chronic renal disease.

51. The method of claim 39 wherein the subject is a mammal.

52. The method of claim 51 wherein the mammal is a human.

53. A method of reducing the activity of a hyperproliferative skin cell, comprising administering to a subject a compound of claim 1, such that reduction of the hyperproliferative skin cell activity occurs.

54. A method of ameliorating a deregulation in the activity of a parathyroid cell, comprising administering to a subject a therapeutically effective amount of a compound of claim 1 so as to ameliorate the deregulation of the parathyroid cell activity.

55. A method of ameliorating a deregulation of calcium and phosphate metabolism, comprising administering to a subject a therapeutically effective amount of a compound of claim 1, so as to ameliorate the deregulation of the calcium and phosphate metabolism.

56. The method of claim 55 wherein the deregulation of the calcium and phosphate metabolism leads to osteoporosis.

57. A method of preventing neuronal loss by contacting a vitamin D₃-responsive neuronal cell with a compound of claim 1, so as to prevent or retard neuron loss.

58. A method of modulating the activity of a vascular smooth muscle cell by contacting a vitamin D₃-responsive smooth muscle cell with a compound of claim 1 so as to modulate the activity of the cell.

59. A pharmaceutical composition comprising a therapeutically effective amount of a compound of claim 1 and a pharmaceutically acceptable carrier.

60. The composition of claim 59 which is suitable for topical administration.

61. The composition of claim 59 which is suitable for oral administration.

62. A packaged compound, comprising a compound of claim 1 packaged with instructions for use of the compound for treating a disorder characterized by an aberrant activity of a vitamin D₃-responsive cell.
